(19) United States
(12) Patent Application Publication
(10) Pub. No.: US 2008/0248567 A1
(43) Pub. Date: Oct. 9, 2008

(54) Methods and products for embryonic stem cell culture

(75) Inventor: Dinender Kumar Singla, Orlando, FL (US)

Correspondence Address: WOLF GREENFIELD & SACKS, P.C., 600 ATLANTIC AVENUE, BOSTON, MA, 02210-2206, US

(73) Assignee: University of Vermont and State Agricultural College, Burlington, VT (US)

(21) Appl. No.: 12/002,971

(22) Filed: Dec. 19, 2007

Related U.S. Application Data
(60) Provisional application No. 60/876,843, filed on Dec. 22, 2006.

(51) Int. Cl. C12N 5/06

(52) U.S. Cl. 435/366; 435/383; 435/377; 435/404

(57) ABSTRACT

The invention relates to methods and media for preparing and maintaining self-renewing pluripotent embryonic stem cells. The methods include, in some embodiments, culturing embryonic stem cells in culture medium that includes culture medium conditioned by cells that express wnt3a.

### RELATED APPLICATION

This application claims the benefit under 35 U.S.C. § 119(e) of U.S. provisional application Ser. No. 60/876,843, filed Dec. 22, 2006, the disclosure of which is incorporated by reference herein in its entirety.

### FIELD OF THE INVENTION

The invention relates to methods and products for culturing self-renewing pluripotent embryonic stem cells. The methods include, in part, the use of culture medium conditioned by wnt3a-expressing cells, and are useful for maintaining self-renewing pluripotent embryonic stem cells in culture.

### BACKGROUND OF THE INVENTION

Embryonic stem (ES) cells are pluripotent and have been identified as a potential source of cells that can be selectively differentiated into cells of specific lineages for use in research and in clinical applications. ES cells are derived from the inner cell mass (ICM) of the pre-implantation embryo and are capable of self renewal (proliferation without differentiation) as well as differentiation into all three embryonic germ layers; ectoderm, endoderm, and mesoderm [Kumar, D., et al., Coron. Artery Dis. 16:111-116 (2005); D. K. Singla & B. E. Sobel, Biochem. Biophys. Res. Commun. 335:637-642 (2005)]. Growth factors in cell cultures influence ES cells to undergo self renewal or differentiation [Kumar, D. et al., Coron. Artery Dis. 16:111-116 (2005); Singla, D. K. & B. E. Sobel, Biochem. Biophys. Res. Commun. 335:637-642 (2005); Behfar, A. et al., FASEB J. 16:1558-1566 (2002)]. The elucidation of growth factors and cytokines contributing to ES cell self-renewal, pluripotency, and differentiation is of considerable interest with respect to ES cell potential for applications in regenerative medicine.

The originally isolated mouse ES cells were propagated indefinitely and maintained pluripotency when grown in the presence of serum and in co-culture with a layer of mitotically inactive mouse embryonic fibroblasts, Buffalo rat liver cell line and STO cells. (Amano et al. Biol Pharm Bull. 2006 August; 29(8):1747-50; Smith and Hooper, Dev Biol. 1987 May; 121(1):1-9) The essential growth factor secreted by these cell lines to maintain ES cells in propagation without differentiation indefinitely was found to be Leukemia Inhibitory factor (LIF). (Ogawa et al. Biochem Biophys Res Commun. 2006 Apr. 28; 343 (1):159-66). Previously, ES cells have been maintained by their culture in the presence of mouse embryonic fibroblasts or LIF-containing medium that allowed them to retain their totipotential capacity. Such maintained cells are totipotent and thus are able to generate cells of all three germ layers (ectoderm, endoderm and mesoderm), after injecting the cultured ES cells into immunocompromised mice in vivo or generating EBs, in vitro. [Yamane et al. Proc Natl Acad Sci USA. 2005 Mar. 1; 102(9):3312-7. D. K. Singla & B. E. Sobel, Biochem. Biophys. Res. Commun. 335:637-642 (2005)].

Wnt/Wg genes related to wingless in Drosophila have been identified in mouse and found to encode glycoproteins that play a role in embryogenesis and postembryonic development. (H. Clevers, Cell. 2006 Nov. 3; 127(3):469-80) The wnt protein family includes proteins that are cysteine-rich lipid-modified glycoproteins that play various roles in embryonic development, apoptosis, axial polarity, cancer, cell proliferation, self-renewal, and differentiation. [Nusse, R., Development 130:5297-5305 (203); Yang, Y., Birth Defects Res. C. Embryo. Today 69:305-317 (2003)]. Wnt3a has been shown to promote proliferation and to suppress osteogenic differentiation of mesenchymal stem cells, and has also been shown to support self-renewal of hematopoietic stem cells. [Reya, T. et al, Nature 423:409-414 (2003).]

Efforts are ongoing to understand these growth factors roles in ES cell self-renewal, pluripotency and differentiation because of the potential applications for ES cells in stem cell therapy applications in regenerative medicine.

### SUMMARY OF THE INVENTION

The invention disclosed herein, in part, includes novel methods of preparing and maintaining ES cell cultures of self-renewing pluripotent embryonic stem cells. Novel methods of preparing and maintaining embryonic stem cells in culture medium comprising culture medium conditioned with wnt3a cells have been identified and it has now been discovered that ES cells grown and prepared using such methods and media retain their status as self-renewing pluripotent ES cells. The invention, in part, also relates to cell culture media that includes medium conditioned by cells that express wnt3a. It has now also been discovered that the preparation and growth of ES cells in cell culture medium that includes wnt3a conditioned medium permits the passaging of the ES cells and the preparation of ES cell culture with self-renewing pluripotent ES cells. Such cells and cell cultures that include self-renewing pluripotent ES cells can be maintained and/or used for regenerative therapeutics and/or research. ES cell cultures prepared using the methods and media of the invention may be used in research and therapeutics and may also be used as a source of cells for differentiation into cells of selected lineage. Newly identified methods and media of the invention obviate the need for feeder cell layers and additional defined media for preparing and/or maintaining cultures of self-renewing pluripotent ES cells.

According to one aspect of the invention, methods of maintaining a self-renewing pluripotent embryonic stem cell culture are provided. The methods include culturing one or more pluripotent embryonic stem cells in culture medium that includes medium conditioned by cells that express wnt3a, under condition suitable for growth and proliferation of the self-renewing pluripotent embryonic stem cell culture. In certain embodiments, the cells that express wnt3a are L3 cells. In some embodiments, the self-renewing, pluripotent embryonic stem cells are mouse self-renewing pluripotent embryonic stem cells. In some embodiments, the self-renewing, pluripotent embryonic stem cells are human self-renewing pluripotent embryonic stem cells. In certain embodiments, the culture medium is from about 0.1% to about 100% (v/v) medium conditioned by cells that express wnt3a. In some embodiments, the culture medium is from about 10% to about 100% (v/v) medium conditioned by cells that express wnt3a. In some embodiments, the culture medium is from about 40% to about 70% (v/v) medium conditioned by cells that express wnt3a. In some embodiments, the culture medium is at least about 50% (v/v) medium conditioned by cells that express wnt3a. In certain embodiments, the pluripotent embryonic stem cells are cultured in the absence of feeder cells. In some embodiments, the culture medium does not comprise leukemia inhibitory factor (LIF). In some embodiments, culturing the self-renewing pluripotent stem cell culture includes passaging the self-renewing pluripotent embryonic stem cell culture. In certain embodiments, the self-renewing pluripotent embryonic stem cell culture is passaged at least 1, 2, 3, 4, 5, 6, 7, 8, or more passages. In some embodiments, culturing the self-renewing pluripotent stem cell culture includes freezing and thawing the self-renewing pluripotent embryonic stem cell culture. In certain embodiments, the self-renewing pluripotent embryonic stem cell culture is maintained at least 3 days. In some embodiments, the self-renewing pluripotent embryonic stem cell culture is maintained at least 5 or more days. In some embodiments, the self-renewing pluripotent embryonic stem cell culture is maintained at least 10 or more days. In certain embodiments, the self-renewing pluripotent embryonic stem cell culture is maintained at least 15 or more days. In some embodiments, the self-renewing pluripotent embryonic stem cell culture is maintained at least 20 or more days. In some embodiments, the self-renewing pluripotent embryonic stem cell culture is maintained at least 40 or more days.

According to another aspect of the invention, methods of maintaining a self-renewing pluripotent embryonic stem cell culture are provided. The methods include culturing one or more pluripotent embryonic stem cells in culture medium comprising wnt3a or a functional variant thereof, under conditions suitable for growth and proliferation of the self-renewing pluripotent embryonic stem cell culture. In some embodiments, the wnt3a is recombinant wnt3a. In certain embodiments, the wnt3a is wnt3a isolated from cells that express wnt3a. In some embodiments, the wnt3a is wnt3a isolated from culture medium conditioned by cells that express wnt3a cells. In some embodiments, the cells that express wnt3a are L3 cells. In certain embodiments, the culture medium includes at least 100 ng/ml wnt3a. In some embodiments, the culture medium includes at least 40 ng/ml, 50 ng/ml, 100 ng/ml, 150 ng/ml, 200 ng/ml, 300 ng/ml, 400 ng/ml, 500 ng/ml, 600 ng/ml, 700 ng/ml, 800 ng/ml, 900 ng/ml, 1000 ng/ml, 1100 ng/ml, 1200 ng/ml, 1300 ng/ml, 1400 ng/ml, or 1500 ng/ml of wnt3a. In some embodiments, the self-renewing pluripotent embryonic stem cells are cultured in the absence of feeder cells. In some embodiments, the culture medium does not comprise leukemia inhibitory factor (LIF). In certain embodiments, culturing the self-renewing pluripotent stem cells includes passaging the self-renewing pluripotent embryonic stem cells. In some embodiments, culturing the self-renewing pluripotent stem cells includes freezing and thawing the self-renewing pluripotent embryonic stem cells.

According to yet another aspect of the invention, isolated, self renewing, pluripotent embryonic stem (ES) cell cultures prepared using any of the aforementioned embodiments or aspects of the invention are provided. In some embodiments, the cells that express wnt3a are L3 cells. In certain embodiments, the self-renewing, pluripotent embryonic stem cells are mouse self-renewing pluripotent embryonic stem cells. In some embodiments, the self-renewing, pluripotent embryonic stem cells are human self-renewing pluripotent embryonic stem cells. In some embodiments, the culture medium is from about 0.1% to about 100% (v/v) medium conditioned by cells that express wnt3a. In some embodiments, the culture medium is from about 10% to about 100% (v/v) medium conditioned by cells that express wnt3a. In certain embodiments, the culture medium is from about 40% to about 70% (v/v) medium conditioned by cells that express wnt3a. In some embodiments, the culture medium is at least about 50% (v/v) medium conditioned by cells that express wnt3a. In some embodiments, the pluripotent embryonic stem cells are cultured in the absence of feeder cells. In certain embodiments, the culture medium does not comprise leukemia inhibitory factor (LIF). In some embodiments, culturing the self-renewing pluripotent stem cell culture comprises passaging the self-renewing pluripotent embryonic stem cell culture. In some embodiments, the self-renewing pluripotent embryonic stem cell culture is passaged at least 1, 2, 3, 4, 5, 6, 7, 8, or more passages. In some embodiments, culturing the self-renewing pluripotent stem cell culture includes freezing and thawing the self-renewing pluripotent embryonic stem cell culture. In certain embodiments, the self-renewing pluripotent embryonic stem cell culture is maintained at least 3 days. In some embodiments, the self-renewing pluripotent embryonic stem cell culture is maintained at least 5 or more days. In some embodiments, the self-renewing pluripotent embryonic stem cell culture is maintained at least 10 or more days. In some embodiments, the self-renewing pluripotent embryonic stem cell culture is maintained at least 15 or more days. In certain embodiments, the self-renewing pluripotent embryonic stem cell culture is maintained at least 20 or more days. In some embodiments, the self-renewing pluripotent embryonic stem cell culture is maintained at least 40 or more days.

According to yet another aspect of the invention, methods of preparing a differentiated embryonic stem cell culture are provided. The methods include maintaining the self-renewing pluripotent embryonic stem cell culture using any method of any of the aforementioned aspects or embodiments of the invention and stimulating differentiation of the self-renewing pluripotent embryonic stem cell culture to prepare a differentiated embryonic stem cell culture. In some embodiments, the differentiated embryonic stem cells are cardiac cells, hepatocytes, or neurons. In certain embodiments, the cells that express wnt3a are L3 cells. In some embodiments, the self-renewing, pluripotent embryonic stem cells are mouse self-renewing pluripotent embryonic stem cells. In some embodiments, the self-renewing, pluripotent embryonic stem cells are human self-renewing pluripotent embryonic stem cells. In certain embodiments, the culture medium is from about 0.1% to about 100% (v/v) medium conditioned by cells that express wnt3a. In some embodiments, the culture medium is from about 10% to about 100% (v/v) medium conditioned by cells that express wnt3a. In some embodiments, the culture medium is from about 40% to about 70% (v/v) medium conditioned by cells that express wnt3a. In certain embodiments, the culture medium is at least about 50% (v/v) medium conditioned by cells that express wnt3a. In some embodiments, the pluripotent embryonic stem cells are cultured in the absence of feeder cells. In certain embodiments, the culture medium does not comprise leukemia inhibitory factor (LIF). In some embodiments, culturing the self-renewing pluripotent stem cell culture includes passaging the self-renewing pluripotent embryonic stem cell culture. In some embodiments, the self-renewing pluripotent embryonic stem cell culture is passaged at least 1, 2, 3, 4, 5, 6, 7, 8, or more passages. In certain embodiments, culturing the self-renewing pluripotent stem cell culture includes freezing and thawing the self-renewing pluripotent embryonic stem cell culture. In some embodiments, the self-renewing pluripotent embryonic stem cell culture is maintained at least 3 days. In certain embodiments, the self-renewing pluripotent embryonic stem cell culture is maintained at least 5 or more days. In some embodiments, the self-renewing pluripotent embryonic stem cell culture is maintained at least 10 or more days. In some embodiments, the self-renewing pluripotent embryonic stem cell culture is maintained at least 15 or more days. In some embodiments, the self-renewing pluripotent embryonic stem cell culture is maintained at least 20 or more days. In certain embodiments, the self-renewing pluripotent embryonic stem cell culture is maintained at least 40 or more days.

According to yet another aspect of the invention, methods of preparing an embryoid body comprising are provided. The methods include culturing a self-renewing pluripotent embryonic stem cell culture maintained by any method of any of the aforementioned aspects or embodiments of the invention under conditions suitable for embryoid body formation. In some embodiments, the cells that express wnt3a are L3 cells. In some embodiments, the self-renewing, pluripotent embryonic stem cells are mouse self-renewing pluripotent embryonic stem cells. In certain embodiments, the self-renewing, pluripotent embryonic stem cells are human self-renewing pluripotent embryonic stem cells. In some embodiments, the culture medium is from about 0.1% to about 100% (v/v) medium conditioned by cells that express wnt3a. In some embodiments, the culture medium is from about 40% to about 70% (v/v) medium conditioned by cells that express wnt3a. In certain embodiments, the culture medium is at least about 50% (v/v) medium conditioned by cells that express wnt3a. In some embodiments, the pluripotent embryonic stem cells are cultured in the absence of feeder cells. In some embodiments, the culture medium does not comprise leukemia inhibitory factor (LIF). In certain embodiments, culturing the self-renewing pluripotent stem cell culture includes passaging the self-renewing pluripotent embryonic stem cell culture. In some embodiments, the self-renewing pluripotent embryonic stem cell culture is passaged at least 1, 2, 3, 4, 5, 6, 7, 8, or more passages. In certain embodiments, culturing the self-renewing pluripotent stem cell culture includes freezing and thawing the self-renewing pluripotent embryonic stem cell culture. In some embodiments, the self-renewing pluripotent embryonic stem cell culture is maintained at least 3 days. In certain embodiments, the self-renewing pluripotent embryonic stem cell culture is maintained at least 5 days. In some embodiments, the self-renewing pluripotent embryonic stem cell culture is maintained at least 10 days. In some embodiments, the self-renewing pluripotent embryonic stem cell culture is maintained at least 20 or more days. In certain embodiments, the self-renewing pluripotent embryonic stem cell culture is maintained at least 40 or more days.

According to yet another aspect of the invention, methods for identifying in cell culture medium conditioned by cells expressing wnt3a, a component that modulates pluripotency and/or self renewal in an embryonic stem cell are provided. The methods include separating one or more components of cell culture medium conditioned by cells that express wnt3a, contacting one or more self-renewing, pluripotent embryonic stem cells with the one or more separated components, culturing the one or more embryonic stem cells under conditions suitable for growth and proliferation of a self-renewing, pluripotent embryonic stem cell, and detecting the presence or absence of an effect on pluripotency and/or self renewal of the embryonic stem cell, wherein the presence of an effect identifies the one or more separated components as modulating pluripotency and/or self renewal of the embryonic stem cell cells. In some embodiments, the cells that express wnt3a are L3 cells. In some embodiments, the self-renewing, pluripotent embryonic stem cells are mouse self-renewing pluripotent embryonic stem cells. In some embodiments, the self-renewing, pluripotent embryonic stem cells are human self-renewing pluripotent embryonic stem cells. In certain embodiments, the pluripotent embryonic stem cells are cultured in the absence of feeder cells. In some embodiments, culturing includes passaging the self-renewing pluripotent embryonic stem cell culture. In some embodiments, culturing the self-renewing pluripotent stem cell culture comprises freezing and thawing the self-renewing pluripotent embryonic stem cell culture. In some embodiments, the self-renewing pluripotent embryonic stem cell culture is maintained at least 3 days. In certain embodiments, the self-renewing pluripotent embryonic stem cell culture is maintained at least 5 or more days. In some embodiments, the self-renewing pluripotent embryonic stem cell culture is maintained at least 10 or more days.

According to another aspect of the invention, a culture medium for maintaining a self-renewing pluripotent embryonic stem cell culture is provided. The culture medium includes at least 30-50% (v/v) of medium conditioned by cells expressing wnt3a. In some embodiments, the cells that express wnt3a are L3 cells. In certain embodiments, the culture medium is at least about 50% (v/v) medium conditioned by cells that express wnt3a. In some embodiments, the culture medium comprises DMEM.

According to yet another aspect of the invention, a conditioned medium for maintaining a self-renewing pluripotent embryonic stem cell culture, the conditioned medium including at least about 50-200 ng/ml wnt3a, is provided.

According to yet another aspect of the invention, culture media that comprises medium conditioned with cells that express wnt3a are provided. Culture media of the invention can be used to prepare and maintain one or more self-renewing pluripotent embryonic stem cells using any of the foregoing aspects of the invention.

According to another aspect of the invention, products formed by any of the foregoing aspects of the invention are provided.

The use of the foregoing self-renewing pluripotent ES cell made using the methods of the invention in the preparation of a medicament, particularly a medicament for treatment of a disease or disorder is also provided.

These and other objects of the invention will be described in further detail in connection with the detailed description of the invention.

Each of the limitations of the invention can encompass various embodiments of the invention. It is, therefore, anticipated that each of the limitations of the invention involving any one element or combination of elements can be included in each aspect of the invention. This invention is not limited in its application to the details of construction an the arrangement of components set forth in the following description or illustrated in the drawings. The invention is capable of other embodiments and of being practiced or o being carried out in various ways.

### BRIEF DESCRIPTION OF THE DRAWINGS

FIG. 1 shows four graphs illustrating the dose-dependent effect of conditioned medium from wnt3a, wnt11, and control cell lines (L cells and NIH3T3) on ES cell proliferation and differentiation. The results are plotted as a percentage change in ES cell compact colonies from D1 to D5. (FIGS. 2A-D) ^{#}p=NS (non-significant) vs wnt3a (50%); −p<0.05 vs wnt3a (10-40% at D4 and D5); *p<0.05 vs L cells (10-50%). (FIGS. 2C, 2D) *p<0.05 vs wnt11 and NIH3T3 (10-50%).

FIG. 2 shows a histogram illustrating the effects of MEK inhibitor, PD098059 and p38 MAPK inhibitor SB203580 on colony formation of ES cells maintained in wnt3a conditioned medium and LIF-containing medium. Approximately 1×10⁴ ES cells were plated in a 60-mm dish and cultured in ES cell medium with or without MAPK inhibitors. Control conditions exposed cells to dimethyl sulfoxide (DMSO) because this vehicle was used to deliver the MAPK inhibitors that were dissolved in DMSO. Colonies were counted 5 days later. The results are plotted as percentage change in ES cell compact colonies. *p<0.05 vs 50% wnt3a; #p<0.05 vs LIF.

### DETAILED DESCRIPTION OF THE INVENTION

The invention, in part, includes methods of preparing and maintaining embryonic stem cells that support self-renewal and pluripotency of the ES cells. Self-renewal is the process by which a stem cell divides to generate daughter stem cells, each of which have developmental potentials that are indistinguishable from the developmental potential of the mother cell. Self-renewal (proliferation without differentiation) processes of ES cells may be involved in embryonic development, maintenance of adult tissues, and may have applications in cell therapy. Pluripotent status of an ES cell means the cell retains the ability to differentiate into a cell type of any of the three embryonic germ layers; ectoderm, endoderm, and mesoderm.

Methods of the invention allow preparation, expansion, and maintenance of ES cells in a self-renewing pluripotent state. Such pluripotent cell culture can be used as a source for generating cells of specific cell lineage for use in research, clinical applications, etc. Unlike previous methods of maintained pluripotent ES cells, the methods of the invention permit long-term maintenance of pluripotent self-renewing ES cells without the need for feeder layers of cells or complex defined media. The invention, in part, also relates to cell culture media that includes medium conditioned by cells that express wnt3a. The methods and media of the invention allow the expansion and maintenance of pluripotent and self-renewing cell cultures that can be a source of cells for differentiation into various cell types useful for research and therapeutic applications.

Methods of the invention are advantageous in that they allow expansion and maintenance of self-renewing pluripotent ES cells in short- and long-term culture. Using methods and media of the invention to grow ES cells in the presence of culture medium that comprises culture medium conditioned by wnt3 a cells, self-renewing pluripotent cells ES cell culture can be produced, expanded, and maintained. Expanded ES cell cultures, prepared using methods and compositions of the invention, can be repeatedly passaged and maintained for extended periods of time. In addition, cells prepared using methods and/or media of the invention can be cultured, harvested, frozen, and re-cultured and thus are suitable for long-term storage and maintenance. Pluripotent self-renewing ES cells and cell cultures prepared using methods and/or media of the invention may be readily utilized for further expansion and/or as a source of founder cells to prepare cell cultures having cells with specific cell lineages. By contacting self-renewing pluripotent cells from ES cell cultures prepared using the methods of the invention with factors that trigger differentiation of pluripotent cells into specific lineages, it is possible to selectively prepare ES cells and cell culture having a desired lineage.

Methods of the invention, in part, relate to the culture of self-renewing pluripotent embryonic stem (ES) cells in medium that includes medium conditioned with cells that express wnt3a protein. As used herein the term “embryonic stem (ES) cells” means mammalian embryonic stem cells. As used herein the term “self-renewing ES cells” means ES cells that proliferate without differentiation. As used herein, the term “pluripotent ES cells” means ES cells that retain the ability to differentiate into cell types of any of the three embryonic germ layers; ectoderm, endoderm, and mesoderm. Embryonic stem cells are originally pluripotent cells that are derived from pre-implantation embryos. Pluripotent ES cells have the capacity to differentiate into any cell type in vivo, and to differentiate into many different cell types in vitro.

Wnt3a protein is a member of the wnt family of proteins. The wnt protein family is a family of cysteine-rich lipid-modified glycoproteins that may be involved in a number of cellular events and developmental mechanisms. In some embodiments of the invention, cell culture medium conditioned with cells that express wnt3a, referred to herein as wnt3a-conditioned medium, can be used to prepare and maintain cultures of self-renewing pluripotent ES cells. As used herein the term “wnt3a-conditioned medium” means culture medium that has been conditioned by cells that express wnt3a. Thus, cells that express wnt3a, e.g., L3 cells or other wnt3a-expressing cells, can be cultured in the presence of culture medium, thereby conditioning the medium. The medium from the culture can be collected and is referred to as conditioned medium. Culture medium in which wnt3a-expressing cells have been cultured is referred to herein as wnt3a-conditioned medium. A non-limiting example of a method of preparing conditioned medium is provided in the Examples section herein. Those of ordinary skill in the art will also be aware of alternative methods that may be used to prepare and harvest (e.g., collect) conditioned media that may be used to prepare and harvest wnt3a-conditioned medium.

Cell culture medium that can be used in the method of the invention, e.g., to prepare wnt3a-conditioned medium and/or to culture ES cells may be any type of culture medium suitable for such purpose. Suitable culture medium may include a basic medium, (e.g. Dulbecco's minimum essential medium [DMEM], etc) and may also include additional factors that are added to the basic medium to provide a suitable medium in which to culture ES cells. As used herein, the term “basic medium” means cell culture medium that has not had additives or supplements added. Examples of basic culture media that may be used in methods and media of the invention, though not intended to be limiting may be DMEM, Eagles MEM, Hams F12, etc. Basis culture media, such as DMEM or other media, which are useful in methods and/or media of the invention may be supplemented with factors that permit or enhance ES cell growth and maintenance. Non-limiting examples of factors or additives that may be added to a basic culture medium are sodium pyruvate, glutamine, penicillin/streptomycin, non-essential amino acids, serum (e.g., fetal bovine, calf serum, human serum, horse serum, etc.) Those of ordinary skill in the art will recognize additional and alternative factors and additives that may be added to a basic culture medium that may be used in methods of the invention.

In some embodiments of the invention, cultured cells may be grown to confluency, which may occur in about 4, 5, 6, 7, 8, 9, 10, or more days. Those of ordinary skill in the art will recognize that the number of days of growth to reach confluent growth may depend on the size of the growth vessel, the number of cells seeded into the vessel, etc. Standard growth procedures may be used to prepare confluent and other cultured cells of the invention.

The invention, in part, involves use of methods to produce a substantially pure culture of self-renewing pluripotent ES cells. As used herein, the word “substantially pure culture” means cells grown in culture that are substantially free of other cultured cell types or cells with non-self-renewing or non-pluripotent status. A substantially pure culture of self-renewing pluripotent ES cells may include from about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% self-renewing pluripotent ES cells.

Using the methods and/or media of the invention, pluripotent self-renewing ES cells may be cultured without the inclusion of a support layer of cells (e.g. 3T3 cells). Support layer cells, which are also known as “feeder cells”, may be mitotically inactivated embryonic fibroblast cells, examples of which are irradiated 3T3 cells or other strain of embryonic fibroblast. In some embodiments of the invention, a layer of support or feeder cells may be present, but the methods of the invention do not require such a support layer. It will be understood by those of ordinary skill in the art, that if a support or feeder layer is present, the mitotically inactivated embryonic cells, e.g. irradiated cells, on which the ES cells are grown, are incapable of proliferation and their presence does not negate the fact that a culture of growing self-renewing and pluripotent ES cells is substantially pure if at least 85%, 90%, 95%, 96%, 97%, 98%, 99%, or 100% of the growing cells in the culture are pluripotent self-renewing ES cells. The presence of only a small percentage or zero percentage of other growing cell types, including ES cells, in a culture of self-renewing pluripotent ES cells means the culture is a substantially pure culture of self-renewing pluripotent ES cells.

The ES cells of the invention can be ES cells obtained from any mammalian species including humans, non-human primates, cats, dogs, sheep, pigs, horses, cows, and rodents such as mice, rats, etc. In some embodiments, the ES cells used in the methods of the invention are mouse ES cells.

ES cells for use in the methods of the invention may be obtained directly from a mammalian pre-implantation embryo, or may be cultured ES stem cells. The ES cells of the invention may be used to prepare ES cell aggregates. As used herein, the term “aggregate” means a group or cluster comprising at least two or more ES cells. ES cell aggregates prepared using methods of the invention, may be clusters or groups of ES cells. Using the methods of the invention ES cells can be used to form ES cell aggregates. An example of an ES cell aggregate, although not intended to be limiting, is an embryoid body.

Embryoid bodies are ES cell aggregates formed in vitro that may be formed using self-renewing pluripotent ES cells generated using methods and/or with media of the invention. Embryoid bodies are three-dimensional groups of ES cells and may include up to several thousand cells aggregated together. Embryoid bodies are routinely used in the art. ES cells generated through methods of the invention can be used to prepare embryoid bodies using art-known methods. An exemplary method of preparing embryoid bodies is provided in the Examples section, and alternative methods will also be known to those of ordinary skill in the art.

Methods of the invention include culturing one or more ES cells under conditions to permit the growth and expansion of ES cells that are pluripotent and self renewing. In some embodiments, one or more ES cells may be placed on a surface adapted for cell attachment. As used herein, the term “adapted for cell attachment” includes surfaces on which the cells will adhere and are suitable for culturing ES cells. Examples of surfaces that may be adapted for cell attachment include, but are not limited to standard tissue culture plates, tubes, and flasks, which generally may have hydrophilic surfaces to enhance adhesion of cells for growth in culture. In some embodiments of the invention, a surface adapted for cell attachment is a surface that has been coated with gelatin (e.g. with a solution of about 0.1% [w/v] gelatin). It will be understood that the shape or form of a surface that is adapted for cell attachment can vary and may include shapes such as tubes, straws, etc. As described above, the ES cells cultured using methods of the invention may be cultured directly on a surface adapted for cell attachment. Thus, growth and culture of ES cells using methods and/or media of the invention may be done in the absence of a layer of feeder or support cells on the surface adapted for cell attachment, although in some embodiments of the invention a feeder or support layer may be present. Using the methods of the invention, ES cells, when placed on a surface adapted for cell attachment, will under appropriate conditions give rise to a cell aggregate such as an embryoid body.

In some embodiments of the invention, self-renewing pluripotent ES cells may be prepared and maintained in liquid cell culture. Liquid cultures of self-renewing pluripotent ES cells may be prepared and maintained using wnt3a-conditioned media in a flask or other container using routine methods of liquid mammalian cell culture. A non-limiting example of a method of liquid cell culture is provided herein in the Examples section.

Methods of the invention, in part, include culturing one or more ES cells under conditions suitable to support self renewal and pluripotency of ES cells such that the ES cells survive in an undifferentiated, pluripotent state and may be maintained and expanded in culture. Methods of the invention include, in part, the culture of ES cells in culture medium that includes wnt3a conditioned medium. For example, wnt3a-conditioned medium may be mixed with non-wnt3a-conditioned medium and used to prepare, culture, and maintain self-renewing pluripotent ES cells. Culture media with which wnt3a-conditioned medium may be mixed may be any suitable ES cell culture medium known in the art. Medium used to mix with the wnt3a-conditioned medium may be the same type of medium used to prepare the wnt3a-conditioned medium or may be a different cell culture medium. The medium may be the same basic culture medium, with the same or different factors and/or supplements added, or may be different basic culture medium with the same or different factors and/or supplements added.

Cell culture media used in the methods of the invention to culture, expand, and maintain self-renewing and/or pluripotent ES cells may be basic or supplemented cell culture medium with a percentage of the total volume of cell culture medium made up of wnt3a-conditioned medium. The percentage of medium conditioned by one or more cells expressing wnt3a that is present in the cell culture medium used to culture one or more ES cells using methods of the invention may range from 0.1% to 100% (v/v). Thus, the percentage of wnt3a-conditioned medium in cell culture medium may be from about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, to about 100%, including all percentages in between each of the listed percentages. In some embodiments, the percentage of wnt3a-conditioned medium in a given volume of cell culture medium is at least about 40%, 50%, 60%, 70%, 80%, 90%, or 95% or more of a total volume of cell culture medium.

Certain factors and growth conditions have previously been recognized in the art as useful to culture self-renewing ES cells. For example, the addition of leukemia inhibiting factor (LIF) has been added to cultures to promote self-renewal of ES cells and ES cells have been cultured on feeder or support cell layers. Using methods and media of the invention such factors and conditions are not necessary to prepare and maintain self-renewing and/or pluripotent ES cells. Thus, in some embodiments, media useful in the methods of the invention may not include exogenous (LIF), as the presence of LIF in the culture medium is not necessary to support self-renewing pluripotent ES cells. Similarly, ES cells may be cultured using methods and/or media of the invention in the presence or absence of a supporting cell layer, e.g. an irradiated 3T3 cell layer or other strain of embryonic fibroblast. The presence of a supporting or feeder layer of cells in not necessary to support self-renewing pluripotent ES cells using the method and/or media of the invention.

In some embodiments of the invention, self-renewing pluripotent ES cells may be prepared, expanded, and/or maintained by culture in cell culture medium (e.g. DMEM) to which natural or recombinant wnt3a has been added in an amount effective to maintain and/or expand ES cells in culture as self-renewing pluripotent ES cells. The amount of wnt3a per ml of culture medium may be least about 40 ng/ml, 50 ng/ml, 60 ng/ml, 70 ng/ml, 80 ng/ml, 90 ng/ml, 100 ng/ml, 110 ng/ml, 120 ng/ml, 130 ng/ml, 140 ng/ml, 150 ng/ml, 160 ng/ml, 170 ng/ml, 180 ng/ml, 190 ng/ml, 200 ng/ml, 210 ng/ml, 220 ng/ml, 230 ng/ml, 240 ng/ml, 250 ng/ml, 260 ng/ml, 270 ng/ml, 280 ng/ml, 290 ng/ml, 300 ng/ml, 310 ng/ml, 320 ng/ml, 330 ng/ml, 340 ng/ml, 350 ng/ml, 360 ng/ml, 370 ng/ml, 380 ng/ml, 390 ng/ml, 400 ng/ml, 410 ng/ml, 420 ng/ml, 430 ng/ml, 440 ng/ml, 450 ng/ml, 460 ng/ml, 470 ng/ml, 480 ng/ml, 490 ng/ml, 500 ng/ml, 510 ng/ml, 520 ng/ml, 530 ng/ml, 540 ng/ml, 550 ng/ml, 560 ng/ml, 570 ng/ml, 580 ng/ml, 590 ng/ml, 600 ng/ml, 610 ng/ml, 620 ng/ml, 630 ng/ml, 640 ng/ml, 650 ng/ml, 660 ng/ml, 670 ng/ml, 680 ng/ml, 690 ng/ml, 700 ng/ml, 710 ng/ml, 720 ng/ml, 730 ng/ml, 740 ng/ml, 750 ng/ml, 760 ng/ml, 770 ng/ml, 780 ng/ml, 790 ng/ml, 800 ng/ml, 810 ng/ml, 820 ng/ml, 830 ng/ml, 840 ng/ml, 850 ng/ml, 860 ng/ml, 870 ng/ml, 880 ng/ml, 890 ng/ml, 900 ng/ml, 910 ng/ml, 920 ng/ml, 930 ng/ml, 940 ng/ml, 950 ng/ml, 960 ng/ml, 970 ng/ml, 980 ng/ml, 990 ng/ml, 1000 ng/ml, 1010 ng/ml, 1020 ng/ml, 1030 ng/ml, 1040 ng/ml, 1050 ng/ml, 1060 ng/ml, 1070 ng/ml, 1080 ng/ml, 1090 ng/ml, 1100 ng/ml, 1110 ng/ml, 1120 ng/ml, 1130 ng/ml, 1140 ng/ml, 1150 ng/ml, 1160 ng/ml, 1170 ng/ml, 1180 ng/ml, 1190 ng/ml, 1200 ng/ml, 1210 ng/ml, 1220 ng/ml, 1230 ng/ml, 1240 ng/ml, 1250 ng/ml, 1260 ng/ml, 1270 ng/ml, 1280 ng/ml, 1290 ng/ml, 1300 ng/ml, 1310 ng/ml, 1320 ng/ml, 1330 ng/ml, 1340 ng/ml, 1350 ng/ml, 1360 ng/ml, 1370 ng/ml, 1380 ng/ml, 1390 ng/ml, 1400 ng/ml, 1410 ng/ml, 1420 ng/ml, 1430 ng/ml, 1440 ng/ml, 1450 ng/ml, 1460 ng/ml, 1470 ng/ml, 1480 ng/ml, 1490 ng/ml, or 1500 ng/ml or more of wnt3a, including every value in between each of the numbers listed. In some embodiments, exogenous wnt3a added to cell culture medium used in methods of the invention, may be added to wnt3a-conditioned medium and in certain embodiments, exogenous wnt3a may be added to cell culture medium that is not wnt3a-conditioned medium.

The invention also involves, in part, the culture and passaging of self-renewing pluripotent ES cells. When a culture of self-renewing pluripotent ES cells has been established, the ES cells can be expanded in culture to form large numbers of self-renewing pluripotent ES cells using any art-known strategies. As used herein the term “expanded” means grown with an increase in cell number. Thus, to expand a cell in culture is to have that cell divide and have more cells produced from that cell and its progeny, forming a culture of substantially identical, self-renewing pluripotent ES cells. Thus, expansion of a self-renewing pluripotent ES cell in a culture may result in an increase in the number of self-renewing pluripotent ES cells in the culture.

Self-renewing pluripotent ES cells in culture may be maintained using procedures known to those of ordinary skill in the art. For example, pluripotent self-renewing ES cells prepared using methods and/or media of the invention may be passaged using standard art-known methods. As used herein, the term “passaging” means, generally, dispersion of cells of a culture, for example, by enzymatic treatment or dilution, followed by replating or subculturing of the dispersed cells onto a new surface (e.g. a new plate) or into a new vessel (e.g. a flask). The cells that are replated and/or sub-cultured are referred to as daughter cells from the prior (parent) culture and using the methods herein, daughter cells are substantially identical to the parent self-renewing pluripotent ES cell.

In some embodiments of the invention, ES cells cultured using methods and/or media of the invention can be passaged 1, 2, 3, 4, 5, 6, 7, 8, 9, 10, 11, 12, 13, 14, 15, 16, 17, 18, 19, 20, or more times. Thus, once a self-renewing pluripotent cell line is established using methods and/or media of the invention, the cells can be propagated for one or more passages without the loss of self-renewing pluripotent properties of the cells. Using methods and/or media of the invention to prepare self-renewing pluripotent ES cells permits culture and/or maintenance of self-renewing pluripotent ES cells for extended periods of time. For example, using methods and media of the invention, pluripotent self-renewing ES cells of the invention may be maintained in propagation for from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100 or more days.

Self-renewing pluripotent cells prepared using methods and/or media of the invention may be frozen and thawed using art-known cell culture methods. Self-renewing pluripotent prepared using the methods and/or media of the invention may be frozen and retain their self-renewing and pluripotent properties upon subculturing under conditions described herein for maintaining self-renewing and/or pluripotent ES cells. For example, after a cell line of self-renewing pluripotent cells has been produced using the methods and/or media of the invention, a stock of the cell line may be frozen. Generally, such cell line stocks can be frozen in liquid nitrogen or at −70° C. Such cells may be frozen slowly, for example at a rate of about a one degree decrease in temperature per minute, and once suitably chilled (e.g., to about −50° C.), the cells can be transferred into liquid nitrogen or to −70° C. In some embodiments, self-renewing pluripotent cells prepared using methods and/or media of the invention may be frozen in the presence of a preservative such as glycerol or dimethyl sulfoxide. A stock of the pluripotent self-renewing cell line can thus be preserved for extended periods of time and cells of the stock may be rapidly thawed, plated, expanded, etc. In some embodiments, self-renewing pluripotent ES cells prepared with methods and/or media of the invention may be maintained in frozen storage and subcultured and passaged repeatedly, essentially allowing the self-renewing pluripotent ES cell line to be retained indefinitely.

Pluripotent self-renewing cells prepared using methods of the invention can be used to generate cells of a specific lineage. As used herein, a cell of “specific lineage” is a cell that differentiates from an ES cell into a cell committed to a particular lineage. For example, a cell that is pluripotent and self-renewing may be grown under conditions suitable to cause the cell to lose its pluripotent and self-renewing status and become committed to a specific cell lineage, e.g, cardiac myocyte lineage, neurons, hepatocytes, kidney cells, dendrocytes, etc.

Self-renewing pluripotent ES cells prepared using methods and/or media of the invention, and ES cells of specific cell lineages derived from ES cell cultures prepared using methods and/or media of the invention may be used to make products. Products that include self-renewing pluripotent ES cells or differentiated ES cells prepared using methods and/or media of the invention may be useful for research and/or therapeutic methods. ES cells prepared using methods and/or media of the invention may be further differentiated for use in research and/or therapeutics or may used in research or may be administered to a subject as self-renewing pluripotent ES cells. Pluripotent self-renewing ES cells as well as cells differentiated from pluripotent self-renewing ES cells generated using the methods and/or media of the invention may be used to treat developmental disorders, cardiovascular disease, endocrine disease, metabolic disease, etc. as well as for treatment of neurodegenerative diseases such as Alzheimer's, disease, Parkinson's disease, etc. and may also be useful for treatment of conditions such as injury, trauma, aging, etc. ES cells prepared and or maintained using methods and/or compositions (e.g., media) of the invention can be used for many purposes including, but not limited to the differentiation of ES cells into any body cell type and use to treat disease. A non-limiting example of such a use is the preparation of cardiac myocytes derived from ES cells that can be used to repair injured myocardium.

Methods of the invention, in part, also include methods to assess wnt3a-conditioned media. Such methods may include the separation of components of wnt3a conditioned media and testing of the separated components either alone or in combination with each other or with other compounds (e.g. growth factors, supplements, etc) to determine their effect on maintaining self-renewing pluripotent cells ES cells in culture. Thus, the invention, in part, includes methods for identifying in wnt3a-conditioned medium (e.g. medium conditioned by wnt3a-expressing cells) one or more components that alone or in combination modulate pluripotency and/or self renewal in ES cells in culture. Such identification and assay methods may include separating one or more components of medium conditioned by cells that express wnt3a, using standard separatory/purifications methods, contacting a pluripotent self-renewing ES cell culture with one or more of the separated components, culturing the ES cell culture under conditions suitable for growth and proliferation of a self-renewing pluripotent, embryonic stem cell culture, and detecting the presence or absence of an effect on pluripotency and/or self renewal of the ES stem cells of the culture. The presence or absence of an effect that results from contact of ES cells with the one or more components may be determined by comparing self-renewing and/or pluripotency status of ES cells contacted with the components, to the level of self-renewal and pluripotency of ES stem cells in a control culture.

Importantly, levels of self-renewing pluripotent ES cells in a culture are advantageously compared to controls according to the invention. A control may be a predetermined value, which can take a variety of forms. It can be a single cut-off value, such as a median or mean. It can be established based upon comparative groups, such as in groups having normal numbers of self-renewing pluripotent ES cells and groups having abnormal amounts of self-renewing pluripotent ES cells. The predetermined value can be arranged, for example, based on a tested population of cells in culture.

The predetermined value, of a course, will depend upon the particular cell culture population selected and the conditions in which the culture is prepared and maintained. For example, a cell population prepared without the presence of factors (e.g., LIF, etc) or cells (e.g. feeder cells) that maintain self-renewal and pluripotency of ES cells will have a different ‘normal’ range than will a population that has been prepared under conditions that permit ES cells to retain self-renewing pluripotent status. Accordingly, the predetermined value selected may take into account the category in which an cell culture falls. Appropriate ranges and categories can be selected with no more than routine experimentation by those of ordinary skill in the art. By statistically significantly higher levels of self-renewing pluripotent cells it is meant high relative to a selected control.

In some embodiments, a control culture may be a culture of ES cells that is prepared and cultured in a substantially identical manner under substantially identical conditions to an ES cell culture to be tested with one or more components of wnt3a-conditioned medium. In some embodiments of the invention, a test culture may be contacted with the component or components to be tested and a substantially identical control culture is not contacted with the component or components and the resulting cultures are compared. The presence or number (e.g. % of total cells) of self-renewing pluripotent ES cells may be determined for each culture and the values compared. A difference in the level or amount of self-renewing and pluripotent cells between the two cultures may indicate an effect of the components on pluripotency and/or self-renewal in ES cells. A determination that the test culture has statistically significantly more self-renewing and/or pluripotent cells than the control culture indicates that the tested component or components enhance(s) maintenance of self-renewal and pluripotency status of ES cells. Using such a method, or other assay methods to detect changes in ES cell cultures following contact with wnt3a-conditioned medium components, cultures contacted with one or more components of the wnt3-conditioned medium in the test culture may be assessed to identify whether or not the wnt3a-conditioned medium component or components has an enhancing effect on the pluripotency and self-renewal status of ES cells in culture.

In cultures, the presence and/or number of self-renewing pluripotent ES cells (e.g. the self-renewing pluripotent status of the ES cells of a culture) may be determined by morphological assessment and/or by other art-known means. A non-limiting example of a method of assessing the presence and/or number of self-renewing pluripotent ES cells in a culture is use of an alkaline phosphatase assay. A description of a alkaline phosphatase assay used to assess pluripotency and self-renewing status of ES cells is described in the Examples section herein. Another non-limiting example of a method that may be used to determine self-renewing pluripotency status of an ES cell or cell culture is histological-based staging for cell markers that correlate with developmental status of an ES cell. Morphological assessment of ES cell status may also be based on morphological appearance of ES cells. Self-renewing and/or pluripotent status in ES cells may be reflected in the growth pattern of ES cells colonies. For example, self-renewing pluripotent ES cell colonies grow in small groups, in contrast to differentiating ES cells, which grow in a more confluent state. Thus, the presence of compact ES cell colonies may be used as an indicator of self-renewing pluripotent status of ES cells.

ES cells that undergo differentiation with growth are not self-renewing or pluripotent may be identified visually as differentiating and/or may be distinguished from self-renewing and pluripotent ES cells using art-recognized methods such as immunohistochemical and/or additional labeling methods. Immunohisotochemical and/or other labeling methods may include the use of antibodies or other agents that differentially bind to and/or label self-renewing pluripotent ES cells and cells that are differentiating or differentiated ES cells. For example, immunohistochemical and/or other labeling methods can be used to detect proteins or other cell components that are present in self-renewing pluripotent cells and not present (or present to a statistically significant lesser extent) in non-self-renewing, non-pluripotent cells. An example of an immunohistochemical assay is provided in the Examples section. Immunohistochemical and/or other labeling methods can be used to detect the expression of proteins or other cell components that are present in non-self-renewing non pluripotent ES cells but not present (or present to a statistically significant lesser extent) in self-renewing pluripotent cells. A protein with a different level of expression in self-renewing pluripotent cells versus non-self-renewing, non-pluripotent ES cells is referred to herein as a “marker protein”.

Immunohistochemical and/or other labeling methods and assays may be performed on test and/or control culture cells using any art-known test to identify marker proteins present in cells or expressed into a cell culture medium. For example, antibody-based labeling of cultured ES cells can be performed to indicate the presence of absence of marker proteins in cultured cells that are only present in differentiating cells and not in self-renewing pluripotent cells. Similarly, antibodies or other binding agents can be used to detect the presence of proteins that are present in self-renewing pluripotent cells and not in differentiating cells. The presence or absence of such marker proteins can be used to determine the self-renewing and/or pluripotent status of ES cells using methods known in the art, including the antibody-based methods. As described herein, the determination of the status of an ES cell as self-renewing and/or pluripotent can be used in assays to identify components of wnt3a-conditioned medium that are effective in maintaining self-renewing and/or pluripotent status of ES cells.

Pluripotent self-renewing ES cells prepared using methods and/or media of the invention can be used to treat diseases and conditions that benefit from cell replacement or augmentation. In some embodiments, a suspension or sheets of pluripotent self-renewing ES cells can be administered to a subject in need of such treatment. In other embodiments, cells differentiated from self-renewing pluripotent ES cells may be administered to a subject in need of such treatments. The application of cells and/or sheets of cells to a subject for treatment is well known in the art. Art-known methods for used for ES cell transfer and administration can be used in conjunction with the methods of the invention described herein. It will be understood that pluripotent self-renewing ES cells or differentiated ES cells prepared using methods and/or media of the invention can be used alone or can be combined with additional cell types, materials. or solutions for administration to a subject for treatment of a disease or condition. For example, in some embodiments, self-renewing pluripotent ES cells prepared using methods and/or media of the invention may be combined with a mesh or other support material for administration to a subject. Those of ordinary skill in the art will understand that additional art-known methods of administering ES cells for therapeutic methods can be used in conjunction with the methods and products of the invention.

It will be understood by those of ordinary skill in the art that any suitable art-known method of detecting the presence and/or amount of nucleic acids that encode a marker protein may also be used to assess marker protein expression—and thus may be used to assess the developmental status of ES cells and in the assessment of components of wnt3a-conditioned medium.

The invention will be more fully understood by reference to the following examples. These examples, however, are merely intended to illustrate the embodiments of the invention and are not to be construed to limit the scope of the invention.

### EXAMPLES

### Example 1

### Introduction

In the present study, we found that conditioned medium prepared from L cells expressing wnt3a can replace feeder cell layers and medium containing LIF in maintaining ES cells in the proliferation without differentiation (self-renewal) state. By contrast, conditioned medium from NIH3T3 cells expressing wnt11 did not. Alkaline phosphatase staining and compact colony formation were used as criteria of cells being in the undifferentiated state. ES cells maintained in medium conditioned by Wnt3a expressing cells underwent freezing and thawing while maintaining properties seen with LIF maintained ES cells. Purified wnt3a did not maintain self-renewal of ES cells for prolonged intervals. Thus, other factors in the medium conditioned by wnt3a expressing cells may have contributed to maintenance of ES cells in a self-renewal state. Pluripotency of ES cells was determined with the use of embryoid bodies in vitro. PD98059, a MEK specific inhibitor, promoted the growth of undifferentiated ES cells maintained in conditioned medium from wnt3a expressing cells. In contrast, the P38 MAPK inhibitor SB230580 did not, suggesting a role for the MEK pathway in self-renewal and differentiation of ES cells maintained in the wnt3a cell conditioned medium. The results indicate that conditioned medium from wnt3a but not wnt11 expressing cells could maintain ES cells in self-renewal and in a pluripotent state.

### Methods

### Cell Lines.

Mouse ES cells (line CGR8) stably transfected with cardiac specific α-actin promoter expressing yellow fluorescence protein (YFP) were studied [Kumar, D., & B. Sun, Biochem. Biophys. Res. Commun. 332:135-141 (2005)]. Mouse L cells and L-wnt3a stably transfected cells were provided by Pierre McCrea (University of Texas). LacZ NIH3T3 cells and NIH3T3-wnt11 stably transfected cells were a gift from Prof. Andreas Kispert (Institut für Molekularbiologie, Germany) [Bergwitz, C. et al., Biochim. Biophys. Acta 1538:129-140 (2001); Vainio, S. et al., Nature 397:405-409 (1999)].

### Preparation of wnt Conditioned Medium.

Mouse L cells, L-wnt3a cells, LacZ NIH3T3 cells, and NIH3T3-wnt11 cells were grown in tissue culture flasks in Dulbecco's minimum essential medium (DMEM) (Gibco, Invitrogen, Carlsbad, Calif.) supplemented with sodium pyruvate, glutamine, penicillin/streptomycin, non-essential amino acids, and 15% fetal bovine serum (Gibco). The wnt3a and wnt11 conditioned medium was prepared as previously described [Pandur, P. et al., Nature 418:636-641 (2002)]. In brief, 2.8×10⁵ cells from L, L-wnt3a, LacZ NIH3T3 or NIH3T3-wnt11 cell lines were grown in T75 flasks containing cell culture medium for 5 days, by which time they had grown to approximately 50% confluence. Medium was replaced and supernatant was collected. After an additional 2 days, when cells had grown to approximately 90% confluence, supernatants were collected again. Both supernatants were pooled, filtered (0.22-μm cellulose syringe filter), and used to assess their effects on growth of ES cells.

### Exposure of ES Cells to wnt Cell Conditioned Medium and MAPK Inhibitors.

ES cells were grown and propagated on gelatin-coated (0.1% gelatin solution, Sigma-Aldrich, St. Louis, Mo.) tissue culture plates and maintained without feeder cells in DMEM as described previously [Kumar, D., & B. Sun, Biochem. Biophys. Res. Commun. 332:135-141 (2005)]. Leukemia inhibitory factor (LIF, 1000 U/ml, Millipore, Billerica, Mass.) was added to the medium to prevent differentiation of ES cells.

ES cells maintained in the LIF medium were trypsinized and distributed at concentrations of 1×10⁴ cells per 60-mm Petri dish with and without LIF. The plates containing ES cells without LIF were supplemented with 10%, 20%, 30%, 40%, and 50% (v/v) concentrations of conditioned medium from either mouse L cells, L-wnt3a cells, LacZ NIH3T3 or NIH3T3-wnt11 cells or with 50 and 100 ng of purified recombinant wnt3a (R&D, Minneapolis, USA). When MEK and MAPK inhibitors were used, ES cells in 50% wnt3a cell conditioned medium or medium containing LIF were supplemented with 12.5 μm PD98059 (Calbiochem) and/or 1 μm SB203580 (Calbiochem) [Burdon, T. et al., Dev. Biol. 210:30-43 (1999); Qi, X., et al., Proc. Natl. Acad. Sci. USA 101:6027-6032 (2004); Yamane, T. et al., Proc. Natl. Acad. Sci. USA 102:3312-3317 (2005)]. The number of compact ES cell colonies, reflecting self-renewal, was determined daily for five days (D1-D5).

### Alkaline Phosphatase Staining.

ES cells maintained with LIF or conditioned medium from L cells, L-wnt3a, LacZ NIH3T3, or NIH3T3-wnt11 cells were rinsed twice with phosphate-buffered saline for 5 min and assayed with the use of alkaline phosphatase kit (Chemicon International, Invitrogen, Billerica, Mass.).

### Embryoid Body Formation and Cell Differentiation.

Embryoid body (EB) formation was assessed as described previously [Singla, D. K. & B. E. Sobel, Biochem. Biophys. Res. Commun. 335:637-642 (2005); Kumar, D., & B. Sun, Biochem. Biophys. Res. Commun. 332:135-141 (2005)]. In brief, ES cells in medium containing LIF or 50% (v/v) wnt3a cell conditioned medium were trypsinized, counted, and cultured in hanging drops (approximately 500 cells per 20-μl drop). They formed EBs in 2 days (D2) that were kept in suspension in differentiating medium for 3 days. Plating was done on gelatin-coated 100-mm tissue culture dishes. ES cells treated with conditioned medium from L, LacZ NIH3T3 or NIH3T3-wnt11 cells did not form EBs. EB formation was assessed for 12 days.

### Immunohistochemical Staining.

EBs derived from ES cells maintained in either LIF or 50% (v/v) L-wnt3a cell conditioned medium were rinsed twice with phosphate-buffered saline (PBS) for 5 min. Cells were fixed and permeabilized with methanol/acetone (2:10) for 20 min at −20° C., incubated with primary antibody against sarcomeric α-actin (1:200) (Sigma-Aldrich), washed, and incubated with secondary antibody Alexa Fluor 568-conjugated with IgM antibody (Molecular Probe—Invitrogen). Nuclei were identified with the nuclear stain Hoechst 33258 (Sigma). Negative controls were performed by incubating cells with primary or secondary antibody alone. Cells were mounted with anti-fade medium (Vector Laboratories, Burlingame, Calif.) and visualized with the use of fluorescence Zeiss microscope.

### Results

To elucidate the effects of medium conditioned by wnt3a and wnt11 expressing cells on self-renewal of ES cells CGR8 mouse ES cells were maintained in self-renewal in medium containing LIF. The results reflect direct effects of wnt and/or effects of other factors elaborated from cells that express wnt. The results demonstrated the morphological appearance of ES cell compact colonies (ES cell colonies growing in small groups) that are known to reflect self-renewal at Day 1, Day 3, and Day 5 following exposure of the cells to 50% (v/v) concentration of wnt3a cell conditioned medium or medium containing LIF. In contrast, ES cells exposed to conditioned medium from either L, NIH3T3, or NIH3T3-wnt11 cells underwent differentiation. Purified wnt3a (100 ng/ml) did not sustain self-renewal for more than 3 days, in contrast to conditioned medium from wnt3a expressing cells, which sustained self-renewal indefinitely. Thus, compact colonies were evident at Day 1 and Day 3 whereas differentiation was observed at Day 5.

The results demonstrated the effects of 50% v/v conditioned medium from LIF, wnt3a, wnt11, control cell lines, and purified recombinant wnt3a (L cells and NIH3T3) on ES cell proliferation and differentiation. Approximately 10⁴ ES cells were plated onto a 60-mm dish and examined daily through day 5. Phase contrast photomicrographs were made of results including images of compact ES cell colonies (small to large) in proliferation without differentiation in LIF containing medium at Day 1, Day 3, and Day 5 and in conditioned medium from wnt3a cells at Day 1, Day 3, and Day 5. ES cells exposed to 50% v/v conditioned medium from control L cells, NIH3T3 or NIH3T3-wnt11 cells showed a mixture of proliferation and differentiation of small colonies at Day 1; small differentiating cells converted into large differentiating cells at Day 3 and Day 5 were observed. Treatment with purified recombinant wnt3a containing medium showed compact colonies suggesting self-renewal up to Day 3 and differentiation at Day 5.

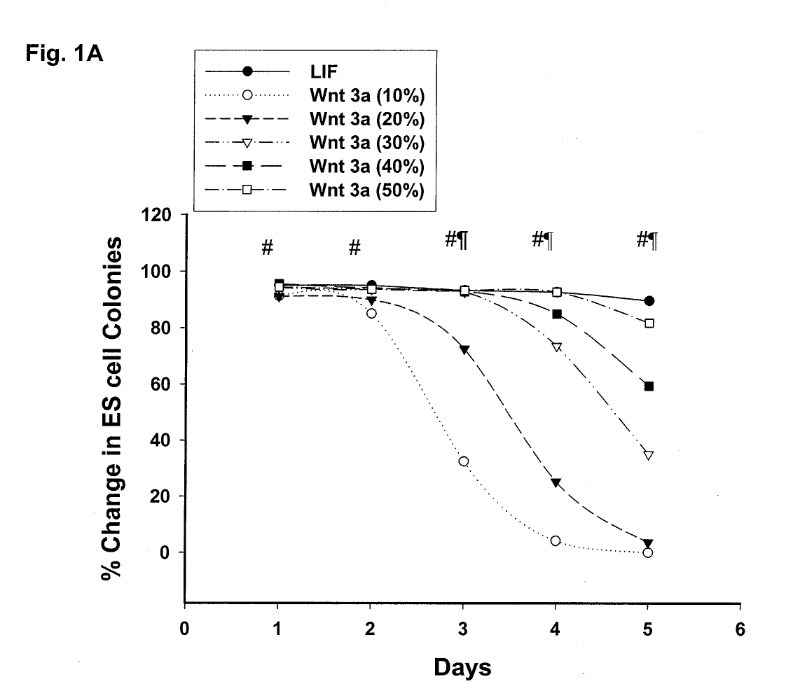

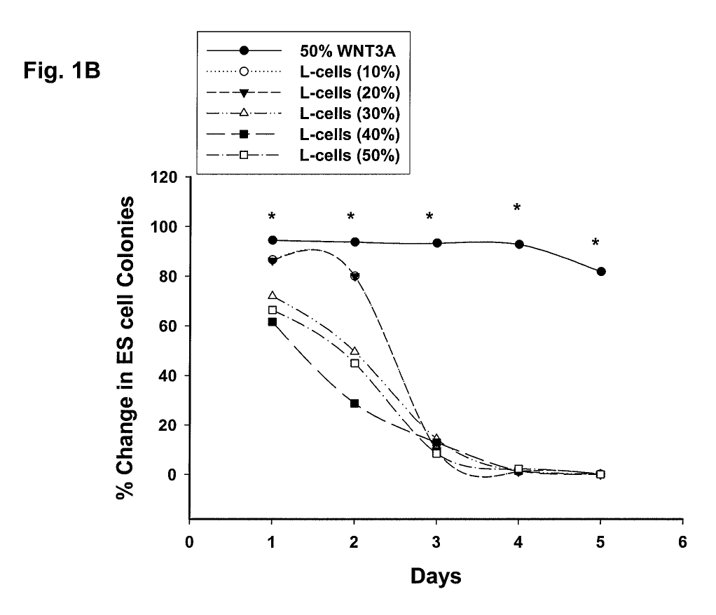

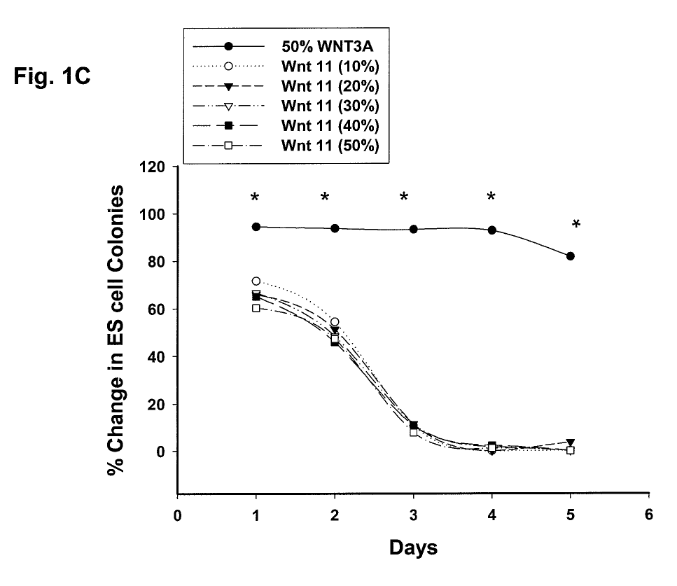

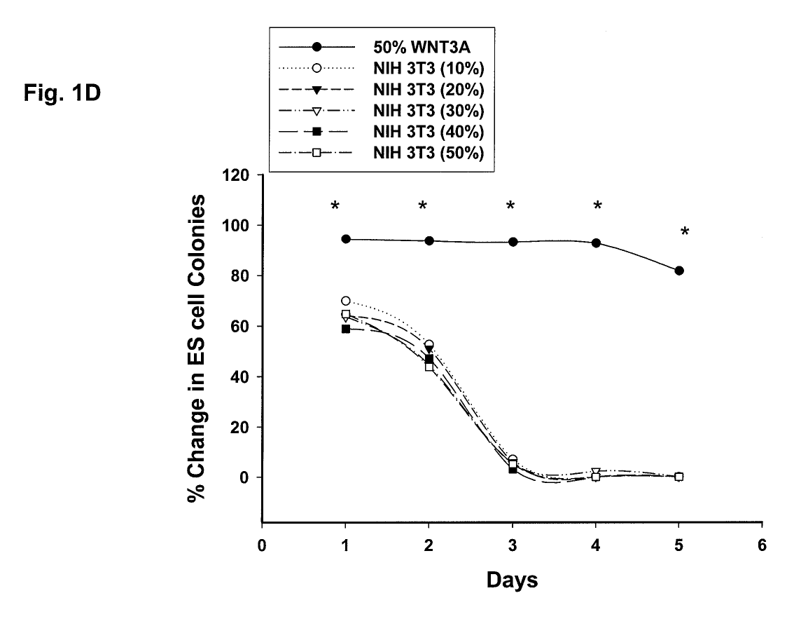

The number of ES cell compact colonies was determined at D1-D5. ES cells exposed to 50% L-wnt3a conditioned medium exhibited 82% compact colonies on D5, significantly greater than the response to conditioned medium from control L cells (FIG. 1B and Table 1, p<0.05). The number of compact colonies associated with exposure to medium from wnt3a cells was not significantly different from that seen with ES cells grown in medium containing LIF, a cytokine used to maintain self-renewal of ES cells (Table 1 and FIG. 1A). By contrast, ES cells exposed to conditioned medium from NIH3T3 or NIH3T3-wnt11 expressing cells did not form compact colonies in numbers similar to those seen with conditioned media from wnt3a cells (Table 1 and FIGS. 1C and D).

TABLE 1
Percent compact ES cell colonies in self-renewal in LIF containing
medium or with conditioned medium from wnts or control cell lines

| | LIF | wnt3a (% conditioned medium v/v) | | | | | L cells ((% conditioned medium v/v) | | | | |
|---|---|---|---|---|---|---|---|---|---|---|---|
| Days | (1000 U/ml) | 10% | 20% | 30% | 40% | 50% | 10% | 20% | 30% | 40% | 50% |
| 1 | 95 ± 1^{#} | 91 ± 4 | 91 ± 4 | 94 ± 1 | 96 ± 1 | 94 ± 2* | 87 ± 3 | 86 ± 7 | 72 ± 4 | 62 ± 15 | 66 ± 3 |
| 2 | 95 ± 1^{#} | 85 ± 4 | 90 ± 5 | 94 ± 1 | 94 ± 1 | 94 ± 1* | 80 ± 4 | 58 ± 6 | 49 ± 4 | 29 ± 3 | 45 ± 4 |
| 3 | 93 ± 2^{#} | 32 ± 15 | 73 ± 2 | 93 ± 1 | 93 ± 0 | 93 ± 2* | 9 ± 2 | 11 ± 2 | 14 ± 2 | 13 ± 3 | 8 ± 1 |
| 4 | 93 ± 3^{#,¶} | 4 ± 4 | 25 ± 6 | 74 ± 1 | 85 ± 4 | 93 ± 1* | 1 ± 1 | 1 ± 1 | 1 ± 2 | 1 ± 2 | 2 ± 3 |
| 5 | 90 ± 1^{#,¶} | 0 | 11 ± 5 | 35 ± 10 | 59 ± 5 | 82 ± 7* | 0 | 0 | 0 | 0 | 0 |

| | wnt11 (% conditioned medium v/v) | | | | | NIH3T3 cells (% conditioned medium v/v) | | | | |
|---|---|---|---|---|---|---|---|---|---|---|
| Days | 10% | 20% | 30% | 40% | 50% | 10% | 20% | 30% | 40% | 50% |
| 1 | 72 ± 4 | 66 ± 4 | 67 ± 7 | 65 ± 9 | 60 ± 9 | 70 ± 5 | 64 ± 3 | 63 ± 2 | 59 ± 1 | 65 ± 8 |
| 2 | 54 ± 4 | 51 ± 3 | 48 ± 1 | 46 ± 1 | 47 ± 1 | 53 ± 3 | 51 ± 4 | 44 ± 3 | 47 ± 1 | 44 ± 3 |
| 3 | 10 ± 3 | 11 ± 8 | 11 ± 3 | 11 ± 4 | 8 ± 4 | 7 ± 1 | 6 ± 8 | 5 ± 2 | 3 ± 1 | 5 ± 1 |
| 4 | 0 | 0 | 2 ± 1 | 2 ± 2 | 1 ± 2 | 0 | 0 | 2 ± 2 | 0 | 0 |
| 5 | 0 | 4 ± 5 | 0 | 19 ± 26 | 0 | 0 | 0 | 0 | 0 | 0 |

Values are expressed as means ± S.E.
^{#}p = NS (non-significant) vs wnt3a (50%).
^{¶}p < 0.05 vs wnt3a (10-40% at D4 and D5).
*p < 0.05 vs L cells or wnt11 or NIH3T3 (10-50%).

To characterize the concentration-dependent effects of conditioned media from wnt or control cells on the formation of compact colonies, ES cells were exposed to 10-50% (v/v) concentration of conditioned medium from wnt or control cells. ES cells exposed to conditioned media from L-wnt3a (30-50%, v/v) expressing cells formed 38-82% compact colonies compared with 0% control L cell conditioned media at D5 (Table 1 and FIG. 1). However, ES cells exposed to L-wnt3a conditioned media [10-20% (v/v)] behaved similarly to those in L cell media (D5, Table 1 and FIGS. 1A and B). ES cells exposed to 10-50% (v/v) of conditioned medium from NIH3T3-wnt11 cells showed no compact colony formation (Table 1 and FIGS. 1C and D).

To determine whether wnt3 a-expressing cells as opposed to wnt11-expressing cells induce self-renewal, alkaline phosphatase staining was used. ES cells cultured with L-wnt3a conditioned medium showed pinkish red staining in compact colonies indicative of cells in proliferation compared with unstained cells cultured with the conditioned medium from L, NIH3T3, and NIH3T3-wnt11 cells or purified wnt3a. However, ES cells grown in LIF medium used as a positive control showed pinkish red staining in compact colonies. To determine whether wnt3a conditioned medium can be used to propagate ES cells in self-renewal longer than 5 days while maintaining their pluripotency on repeated freeze-thaw in liquid N2, ES cells were maintained in propagation for 6-8 passages (approximately 40 days in culture). Unstained ES cells were seen to be differentiated with conditioned medium from control L cells, NIH3T3 or NIH3T3-wnt11 cells and purified recombinant wnt3a, and AP stained cells from these groups showed no pinkish red staining. Note that AP staining was detected as pinkish red coloring in positive cells.

To assess pluripotency of ES cells maintained in conditioned media from wnt3a cells EBs were developed in vitro. Embryoid bodies (EBs) were generated from CGR8 ES cells maintained in 50% wnt3a conditioned medium or LIF containing medium. EB immunostaining for cardiac specific sarcomeric α-actin (red) from wnt3a-conditioned media maintained ES cells showed and from LIF maintained ES cells. Nuclei were stained blue with Hoeschst 33258. EBs were found to synchronously and rhythmically beat and stained with cardiac-specific α-actin consistent with the formation of a mesodermal germ layer. Similar results were obtained with EBs derived from ES cells maintained in LIF containing medium.

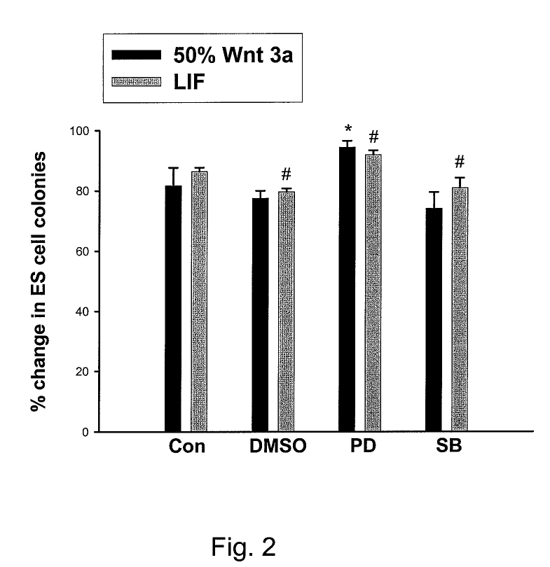

The ERK pathway has been implicated in self-renewal of ES cells maintained in medium containing LIF and bone morphogenic protein-4 (BMP4) [Qi, X., et al., Proc. Natl. Acad. Sci. USA 101:6027-6032 (2004); Yamane, T. et al., Proc. Natl. Acad. Sci. USA 102:3312-3317 (2005)]. However, inhibition of ERK with PD98059 potentiates self-renewal [Qi, X., et al., Proc. Natl. Acad. Sci. USA 101:6027-6032 (2004); Yamane, T. et al., Proc. Natl. Acad. Sci. USA 102:3312-3317 (2005)]. Inhibition of the MEK pathway/ERK with PD98059 led to a modest enhancement of self-renewal by wnt3a conditioned medium (FIG. 2) By contrast, inhibition of p38 MAPK with SB203580 did not affect self-renewal.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A method of maintaining a self-renewing pluripotent embryonic stem cell culture, the method comprising culturing one or more pluripotent embryonic stem cells in culture medium comprising medium conditioned by cells that express wnt3a, under conditions suitable for growth and proliferation of the self-renewing pluripotent embryonic stem cell culture.

2. The method of claim 1, wherein the cells that express wnt3a are L3 cells.

3-4. (canceled)

5. The method of claim 1, wherein the culture medium is from about 0.1% to about 100% (v/v) medium conditioned by cells that express wnt3a.

6-8. (canceled)

9. The method of claim 1, wherein the pluripotent embryonic stem cells are cultured in the absence of feeder cells.

10. The method of claim 1, wherein the culture medium does not comprise leukemia inhibitory factor (LIF).

11-19. (canceled)

20. An isolated, self renewing, pluripotent embryonic stem (ES) cell culture prepared using the method of claim 1.

21. The isolated, self-renewing, pluripotent ES cell culture of claim 20, wherein the cells that express wnt3a are L3 cells.

22-23. (canceled)

24. The isolated, self-renewing, pluripotent ES cell culture of claim 20, wherein the culture medium is from about 0.1% to about 100% (v/v) medium conditioned by cells that express wnt3a.

25-27. (canceled)

28. The isolated, self-renewing, pluripotent ES cell culture of claim 20, wherein the pluripotent embryonic stem cells are cultured in the absence of feeder cells.

29. The isolated, self-renewing, pluripotent ES cell culture of claim 20, wherein the culture medium does not comprise leukemia inhibitory factor (LIF).

30-38. (canceled)

39. A method of preparing a differentiated embryonic stem cell culture, the method comprising, maintaining the self-renewing pluripotent embryonic stem cell culture using the method of claim A1 and stimulating differentiation of the self-renewing pluripotent embryonic stem cell culture to prepare a differentiated embryonic stem cell culture.

40. The method of claim 39, wherein the differentiated embryonic stem cells are cardiac cells, hepatocytes, or neurons.

41. The method of claim 39, wherein the cells that express wnt3a are L3 cells.

42-43. (canceled)

44. The method of claim 39, wherein the culture medium is from about 0.1% to about 100% (v/v) medium conditioned by cells that express wnt3a.

45-48. (canceled)

49. The method of claim 39, wherein the culture medium does not comprise leukemia inhibitory factor (LIF).

50-85. (canceled)

86. A culture medium for maintaining a self-renewing pluripotent embryonic stem cell culture, the culture medium comprising at least 30-50% (v/v) of medium conditioned by cells expressing wnt3a.

87. The culture medium of claim 86, wherein the cells that express wnt3a are L3 cells.

88. The culture medium of claim 86, wherein the culture medium is at least about 50% (v/v) medium conditioned by cells that express wnt3a.

89. The culture medium of claim 86, wherein the culture medium comprises DMEM.

90. A conditioned medium for maintaining a self-renewing pluripotent embryonic stem cell culture, wherein the conditioned medium comprises at least about 50-200 ng/ml wnt3a.
